(19) United States
(12) Patent Application Publication
(10) Pub. No.: US 2026/0014177 A1
(43) Pub. Date: Jan. 15, 2026

(54) ANTIHYPERTENSIVE DRUG AND ANTIHYPERTENSIVE FOOD COMPOSITION

(71) Applicant: Marukyou Suisan Co., Ltd., Wakkanai-shi Hokkaido (JP)

(72) Inventors: Nobuyuki MIYAMOTO, Wakkanai-shi Hokkaido (JP); Naonobu TSUTSUMI, Wakkanai-shi Hokkaido (JP)

(73) Assignee: Marukyou Suisan Co., Ltd., Wakkanai-shi, Hokkaido (JP)

(21) Appl. No.: 18/844,491

(22) PCT Filed: Mar. 7, 2023
(86) PCT No.: PCT/JP2023/008494

§ 371 Date: Mar. 6, 2025

(30) Foreign Application Priority Data
Mar. 8, 2022 (JP) 2022-034976

(51) Int. Cl. A61K 31/702; A61P 9/12

(52) U.S. Cl. CPC A61K 31/702; A61P 9/12

(57) ABSTRACT

[Problem]

To provide a technique for lowering blood pressure with the use of a chondroitin sulfate oligosaccharide.

[Solution]

An antihypertensive drug and an antihypertensive food composition comprising a chondroitin sulfate oligosaccharide as an active ingredient, a method for lowering blood pressure, a method for preventing or improving hypertension, and use of a chondroitin sulfate oligosaccharide for producing an antihypertensive drug. According to the present invention, blood pressure can be lowered or an increase in blood pressure can be inhibited by using the active ingredient by oral administration, namely, a simple and noninvasive form of usage. Thus, the present invention can contribute to the prevention or improvement of hypertensive conditions.

### TECHNICAL FIELD

The present invention relates to an antihypertensive agent and an antihypertensive food composition containing chondroitin sulfate oligosaccharide as an active ingredient, a method for lowering blood pressure, a method for preventing or improving hypertension, and use of chondroitin sulfate oligosaccharide for producing an antihypertensive agent.

### BACKGROUND ART

Chondroitin sulfate (CS) is a type of glycosaminoglycan having a structure in which a sulfate group is bonded to a long sugar chain made of a disaccharide repeating structure of uronic acid and N-acetyl-D-galactosamine. Many sulfuric acid groups cause strongly negative charges, leading to a property rich in water retainability and elasticity. The distribution of CS in vivo is widely observed not only in cartilage but also in various tissues such as connective tissues including skin and brain. CS interacts with cell growth factors and extracellular matrix ingredients to control various cellular activities such as cell adhesion, migration, proliferation, differentiation, and morphogenesis, and is known to play an important role in cushioning action particularly in cartilage.

In addition, Patent Literature 1 discloses a diastolic blood pressure increase inhibitor containing a CS-containing porcine cartilage extract as an active ingredient.

### CITATIONS LIST

### Patent Literature

Patent Literature 1: Japanese Patent No. 6629469

### SUMMARY OF INVENTION

### Technical Problems

As described above, it has been known that the CS-containing porcine cartilage extract suppresses an increase in diastolic blood pressure, but it has not been known that a chondroitin sulfate oligosaccharide (CS oligosaccharide) obtained by reducing the molecular weight of CS lowers the blood pressure.

The present inventors have successfully developed a technique for producing a CS oligosaccharide by hydrolyzing CS under high-temperature and high-pressure conditions to reduce the molecular weight of CS (Japanese Patent No. 6146733). As a result of intensive studies, the present inventors have found that the CS oligosaccharide reduces blood pressure.

That is, the present invention has been made to solve the above problems, and an object of the present invention is to provide a technique for lowering blood pressure using a CS oligosaccharide. More specifically, an object of the present invention is to provide an antihypertensive agent and antihypertensive food composition containing a CS oligosaccharide as an active ingredient, a method for reducing blood pressure, a method for preventing or improving hypertension, and use of a CS oligosaccharide for producing an antihypertensive agent.

### Solutions to Problems

(1) The antihypertensive agent according to the present invention contains a CS oligosaccharide as an active ingredient.

(2) The antihypertensive agent according to the present invention may be used for lowering systolic blood pressure.

(3) In the antihypertensive agent according to the present invention, the CS oligosaccharide may have 2 to 38 sugar residues as constituent saccharides.

(4) The antihypertensive food composition according to the present invention contains a CS oligosaccharide as an active ingredient.

(5) The antihypertensive food composition according to the present invention may be used for reducing systolic blood pressure.

(6) In the antihypertensive food composition according to the present invention, the CS oligosaccharide may have 2 to 38 sugar residues as constituent saccharides.

(7) The method for reducing blood pressure according to the present invention includes a step of administering a CS oligosaccharide to a human or animal to reduce the blood pressure of the human or animal. From the viewpoint of industrial applicability, the present method can exclude medical effects or medical practice.

(8) The method for preventing or improving hypertension according to the present invention includes a step of administering a CS oligosaccharide to a human or animal who has developed or is at risk of developing hypertension to reduce the blood pressure of the human or animal.

(9) The use according to the present invention is the use of a CS oligosaccharide for producing an antihypertensive agent.

### Advantageous Effects of Invention

Hypertension is typically often asymptomatic until a complication occurs in the target organ, but untreatment of severe or prolonged hypertension would cause significant complications. Examples of the organs impaired by hypertension include brain, eye, heart, and kidney, and examples of the complications include hypertensive encephalopathy, hypertensive crisis, heart attack, heart failure, myocardial infarction, arrhythmia, stroke, cerebral hemorrhage, cerebral infarction, vascular dementia, renal failure, nephrosclerosis, arteriosclerosis, aneurysm, and fundus hemorrhage (Merck & Co., Inc., Kenilworth, NJ, USA, MSD Manual Home Edition, 06. Heart and Blood Vessel Diseases, hypertension, author: George L. Bakris, final review/revision date: October 2019, [searched on Mar. 2, 2023], Internet <URL: https://www.msdmanuals.com/ja-jp/%E3%83%9B&E3%83%BC%E3%83%A0/06-%E5%BF%83%E8%87%93%E3%81%A8%E8%A1%80%E7%AE%A1%E3%81%AE%E7%97%85%E6%B0%97/%E9%AB%98%E8%A1%80%E5%9C%A7/%E9%AB98%E8%A1%80%E5%9C%A7?query=E9%AB%98%E8%A1%80%E5%9C%A7#v718009_ja>) (National Research and Development Corporation National Cardiovascular Disease Research Center, National Cardiovascular Disease Research Center Hospital TOP>To Patients>To Know About Cardiovascular Disease>About Diseases>Hypertension, Literature: Yuhei Kono, Last Updated: Sep. 21, 2021 [searched on Mar. 2, 2023], Internet <URL: https://www.ncvc.go.jp/hospital/pub/knowledge/disease/hyper tension-2/#:˜:text=%E9%AB%98%E8%A1%80%E5%9C%A7%E7%97%87%E3%81%A8%E3%81%AF%E3%80%81%E3%81%8F%E3%82%8A%E8%BF%94%E3%81%97,%E9%AB%98%E8%A1%80%E5%9C%A7%E3%81%A8%E8%A8%BA%E6%96%AD%E3%81%95%E3%82%8C%E3%81%BE%E3%81%99%E3%80%82>). For this reason, it is important to maintain the blood pressure within an appropriate value range. The present invention is expected to lower the blood pressure or suppress an increase in the blood pressure to allow contributing to prevention or improvement of the hypertension state. In addition, the present invention is expected to lower the blood pressure or suppress an increase in the blood pressure, thereby allowing to contribute to prevention or improvement of the above-described complications caused by hypertension.

In addition, the CS oligosaccharide as an active ingredient can exhibit an antihypertensive effect by oral ingestion as shown in Examples described later. Thus, according to the present invention, the active ingredient can be used in the form of oral ingestion, which is a simple and non-invasive form of use, to lower blood pressure or suppress an increase in blood pressure.

Further, as shown in the Examples below, the CS oligosaccharide exerts particularly effectively an antihypertensive effect when the blood pressure is high. For this reason, the present invention can be used safely without concerning about excessive reduction in blood pressure.

### BRIEF DESCRIPTION OF DRAWINGS

FIG. 1 is a structural formula showing a disaccharide CS oligosaccharide (CS2). The left side is CS2 with a saturated structure at the non-reducing end, and the right side is CS2 with a double bond at C4-C5 of the non-reducing end.

FIG. 2 is a schematic diagram showing an oligosaccharide production apparatus used in the examples.

FIG. 3 is a line graph showing the relationship between the number of weeks since the start of ingestion of the test food or placebo food and systolic blood pressure in the test food group and the placebo food group.

FIG. 4 is a line graph showing the relationship between the number of weeks since the start of ingestion of the test food or placebo food and diastolic blood pressure in the test food group and the placebo food group.

FIG. 5 is a line graph showing the relationship between the number of weeks since the start of take of the test food or placebo food and systolic blood pressure in the high blood pressure class of the test food group and the placebo food group.

FIG. 6 is a line graph showing the relationship between the number of weeks since the start of take of the test food or placebo food and diastolic blood pressure in the high blood pressure class of the test food group and the placebo food group.

FIG. 7 is a line graph showing the relationship between the number of weeks since the start of ingestion of the test food or placebo food and systolic blood pressure in the normal blood pressure class of the test food group and the placebo food group.

FIG. 8 is a line graph showing the relationship between the number of weeks since the start of take of the test food or placebo food and diastolic blood pressure in the normal blood pressure class of the test food group and the placebo food group.

The present invention will be described in detail below. The present invention provides an antihypertensive agent, an antihypertensive food composition, a method for lowering blood pressure, a method for preventing or improving hypertension, and use of a CS oligosaccharide for producing an antihypertensive agent.

The antihypertensive agent refers to an agent that has the effect of lowering blood pressure, or an agent that is used for the purpose of bringing about such an effect in the living body.

The antihypertensive food composition refers to a food composition that has the effect of lowering blood pressure, or a food composition that is used for the purpose of bringing about such an effect in the living body.

In the present invention, “lowering blood pressure” includes: (i) lowering blood pressure after ingestion of a CS oligosaccharide compared to before ingestion; (ii) increasing the degree of decrease in blood pressure in the ingestion group as compared with the absence of the CS oligosaccharide (non-ingestion group) with the ingestion of the CS oligosaccharide (ingestion group) even if blood pressure decreases over time in both groups; and (iii) decreasing the degree of increase in blood pressure in the ingestion group as compared with the absence of the CS oligosaccharide with the ingestion group even if blood pressure increases over time in both groups. That is, “lowering blood pressure” may be used interchangeably with “suppressing an increase in blood pressure”.

In the present invention, the CS oligosaccharide refers to one having a structure in which a sulfate group is bound to a sugar chain made of a repeating disaccharide structure of uronic acid and N-acetyl-D-galactosamine (chondroitin sulfate structure), and the number of sugar residues as constituent saccharides (the number of sugar residues) is less than that of chondroitin sulfate. In the present invention, chondroitin sulfate may be referred to as “CS of polymer” or “polymeric CS” to contrast with the CS oligosaccharide.

Herein, the molecular weight and number of sugar residues of the polymeric CS are difficult to precisely determine because these can vary depending on the extraction method from the raw material, but the following has been reported.

The molecular weight of CS is 30,000 to 50,000 (equivalent to approximately 120 to 200 sugar residues) (Chemical Dictionary (2nd edition), ISBN978-4-627-24012-4, Morikita Publishing, December 2009).

The molecular weight of CS is 20,000 (equivalent to approximately 80 sugar residues) (World Encyclopedia, 2nd Edition, edited by Hitachi Digital Heibonsha, ISBN978-4-582-04101-9, Heibonsha, October 1998).

The mass of CS derived from bovine tracheal cartilage is 15 kDa (equivalent to approximately 60 sugar residues), and the mass of CS derived from whale cartilage is 21 kDa (equivalent to approximately 80 sugar residues) (Igarashi et al., Effect of Molecular Sizes of Chondroitin Sulfate on Interaction with L-Selectin, International Journal of Carbohydrate Chemistry, Vol. 2013, Article ID 856142, May 8, 2013).

The mass of CS derived from ray fish cartilage is 124 kDa (equivalent to approximately 500 sugar residues) (Hashiguchi et al., Demonstration of the hepatocyte growth factor signaling pathway in the in vitro neuritogenic activity of chondroitin sulfate from ray fish cartilage. Biochimica et Biophysica Acta—General Subjects, 1810, 406-413 (2011)).

In addition, according to the product catalog, the molecular weight of commercially available sodium chondroitin sulfate derived from shark (Seikagaku Corporation, compliant with the Japanese Pharmacopoeia Non-Drug Standards) is approximately 20,000 (product grade: ND-K, equivalent to approximately 80 sugar residues) and approximately 30,000 (product grade: GK, equivalent to approximately 120 sugar residues).

From these findings, it can be said that the molecular weight of the polymeric CS is in the range of approximately 15,000 to 124,000, and the number of sugar residues is in the range of approximately 60 to 500.

The number of sugar residues in the CS oligosaccharide may be more than that of monosaccharides, but less than that of the polymeric CS (the number of sugar residues is typically 60 to 500). Specific examples of the number of sugar residues in the CS oligosaccharide include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48.

The CS of polymer is not absorbed through the intestinal tract when ingested orally, but the CS oligosaccharide has low molecular weight and thus has high permeability through the intestinal wall, allowing to be absorbed directly from the intestinal tract into the body when ingested orally (International Application PCT/JP2021/3414, U.S. Pat. No. 6,146,733: paragraphs [0081]-[0086], [FIG. 18], [FIG. 19]). It is considered that the CS oligosaccharide absorbed into the body is transferred to the bloodstream and exerts an antihypertensive effect.

The CS oligosaccharide may be obtained by chemical synthesis or may be obtained by decomposing CS of polymer into smaller molecules by known methods. In addition, the CS oligosaccharide may be commercially available products (for example, “nano-type chondroitin (Marukyou Bio Foods Co., Ltd.)”).

Examples of the method for decomposing CS into smaller molecules include: a method of hydrolysis under high temperature and high pressure conditions (175° C.≤temperature T≤220° C. and 5 MPa≤pressure P≤25 MPa) (Patent Publication No. 6146733); a method of hydrolysis using an acid such as hydrochloric acid (Cifonelli, Carbohydrate Res., Vol. 2, pp. 150-161, 1966); a method of desorption decomposition or hydrolysis using enzymes such as chondroitinase ABC, chondroitinase ACII, testicular hyaluronidase, and CS decomposition enzyme (Patent Publication No. 9-168384); and a method of maintaining a CS aqueous solution with a pH of 2.5 to 12.0 under hydrothermal conditions of less than 100 to 160° C. for less than 5 to 20 minutes (Patent Publication No. 2010-77256).

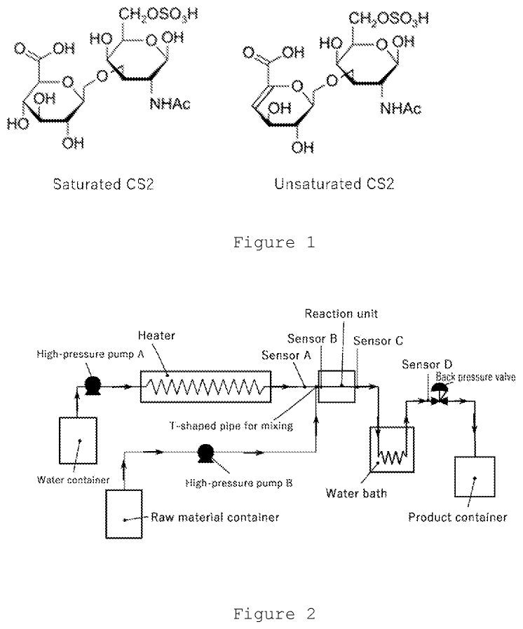

Among the above methods, the hydrolysis method can provide the CS oligosaccharide having a structure in which the non-reducing end is saturated (saturated type). Herein, the saturated type refers to a structure that does not have a double bond between the 4th and 5th carbons (C4-C5) of the uronic acid residue located at the non-reducing end of the oligosaccharide, and an unsaturated type refers to a structure that has a double bond at C4-C5. FIG. 1 shows the saturated and unsaturated structures of a 2 sugar CS oligosaccharide (CS2). The saturated CS oligosaccharide is difficult to be assimilated by intestinal bacteria and can be transported into the blood while maintaining the chondroitin sulfate structure (International Application PCT/JP2021/3414; Example 3 and the like). Therefore, it is considered that the saturated CS oligosaccharide is more easily absorbed by the body.

In addition, the saturated CS oligosaccharide does not absorb ultraviolet light with a wavelength of 240 nm, but the unsaturated CS oligosaccharide absorbs light at this wavelength. Therefore, if a sample is subjected to HPLC and detected with a UV detector at a wavelength of 240 nm, it is possible to determine whether the sample is saturated or unsaturated CS oligosaccharides based on whether a peak is detected.

Examples of the structures that CS has (2-sugar unit structures) include: a glucuronic acid-N-acetyl-D-galactosamine 4-sulfate structure (so-called chondroitin sulfate A structure); a glucuronic acid-N-acetyl-D-galactosamine 6-sulfate structure (so-called chondroitin sulfate C structure); an iduronic acid-N-acetyl-D-galactosamine 4-sulfate structure (so-called chondroitin sulfate B structure, also called dermatan sulfate); a glucuronic acid-2-sulfate-N-acetyl-D-galactosamine 6-sulfate structure (so-called chondroitin sulfate D structure); a glucuronic acid-N-acetyl-D-galactosamine 4,6-sulfate structure (so-called chondroitin sulfate E structure); a glucuronic acid-2-sulfate-N-acetyl-D-galactosamine 4,6-sulfate structure (so-called chondroitin sulfate T structure); and a O structure without a sulfate bond. The bond of the sulfate group is heterogeneous even within a single polymeric CS, and therefore, in many cases, a single polymeric CS is considered to have multiple structures. In the present invention, CS mainly containing any of the above structures can be used.

If CS is decomposed to provide a CS oligosaccharide, the raw material CS may be a commercially available product extracted from animal cartilage or the like. The composition ratio of 2-sugar units in CS contained in animal cartilage (the ratio of 2-sugar units of a specific structure to the total number of 2-sugar units contained in CS) is known to vary depending on the animal from which it is derived. Chondroitin sulfate derived from the cartilage of cartilaginous fish such as rays, sharks, chimaeras, and whale sharks are mainly made of chondroitin sulfate C, and also contains chondroitin sulfates A, D, E, and O. On the other hand, chondroitin sulfate derived from the cartilage of mammals such as cows, whales, rabbits, sheep, and pigs, and birds such as chickens, is mainly made of chondroitin sulfate A and also contains chondroitin sulfates C and O. Chondroitin sulfate derived from the cartilage of mollusks such as squid is mainly made of chondroitin sulfate E, and contains chondroitin sulfates A, C, and O. The 2-sugar unit composition ratio of chondroitin sulfate contained in cartilaginous fish is, for example, chondroitin sulfate C:chondroitin sulfate D=50 to 70:1 to 10, and also chondroitin sulfates O, A, and E are contained.

The 2-sugar unit composition ratio of CS can be quantified by treating CS with chondroitinase to generate unsaturated 2-sugars, separating by high performance liquid chromatography (HPLC), and detecting the peaks corresponding to each 2-sugar unit (A, C, or the like) of CS (refer to JHFA Product Guide: Chondroitin Sulfate Foods, published Aug. 20, 2015, Japan Health and Nutrition Food Association, p. 13-18). In addition, the animal from which CS originates can be identified by “animal-origin DNA testing” or “animal-origin protein testing” based on the DNA or protein remaining in the CS (for example, refer to the Japan Food Research Laboratories, http://www.jfrl.or.jp/item/allergens/post-62.html).

The CS oligosaccharide can be used by being administered to the human or animal body. That is, the present invention also provides a method for lowering blood pressure, which includes a step of lowering the blood pressure of a human or animal by administering a CS oligosaccharide to the human or animal.

Examples of the method for administering a CS oligosaccharide include oral administration, subcutaneous injection, intravenous injection, intramuscular injection, intrathecal injection, sublingual administration, oral mucosal administration, rectal administration, vaginal administration, eye drops, ear drops, nasal administration, oral inhalation, spray inhalation, and transdermal administration.

The CS oligosaccharide exerts an antihypertensive action and thus can be used to produce an antihypertensive agent. Examples of the form of the antihypertensive agent include not only a CS oligosaccharide, which is the active ingredient, but also pharmaceuticals, quasi-drugs, supplements, food additive agents, feed additives, and cosmetics, in which the agent is combined with an appropriate excipient or carrier. Such pharmaceuticals, quasi-drugs, supplements, food additive agents, feed additives, and cosmetics can be produced by methods known to those skilled in the art.

The contents of the CS oligosaccharide in pharmaceuticals, quasi-drugs, supplements, food additive agents, and feed additives vary depending on the dosage form, but can be set to, for example, 0.001 to 90% by mass, 0.01 to 85% by mass, or 0.1 to 80% by mass (measured by HPLC method) based on the dry mass.

In addition, the content of the CS oligosaccharide in cosmetics varies depending on the dosage form, but can be set to, for example, 0.0001 to 80% by mass, 0.001 to 60% by mass, or 0.01 to 50% by mass (measured by HPLC method) based on the dry mass.

The CS oligosaccharide exerts the antihypertensive action and thus can be used to produce an antihypertensive food composition. Examples of the form of the antihypertensive food composition include not only those made of the CS oligosaccharide, which is the active ingredient, but also the form of ordinary food and drink such as confectionery, beverages, processed foods, health foods, and foods for infants. If the form of a food or drink is provided, the production can be performed by adding the active ingredient during the usual production process. In this case, the food composition includes not only food and drink for humans, but also feed, pet food, beverages, and the like (animal feed and the like) for livestock, racehorses, and pet animals. Animal feeds and the like are essentially the same as food and drink, except that they are for animals, and thus the descriptions of food compositions in the present description can be similarly applied to animal feeds and the like.

The content of the CS oligosaccharide in a food composition varies depending on the form of the food and drink, but can be set to, for example, 0.001 to 99% by mass, 0.01 to 80% by mass, or 1 to 80% by mass (measured by HPLC method) based on the dry mass.

The standard amount of administration (intake) of the CS oligosaccharide per serving or per day can be appropriately set according to the product form of the antihypertensive agent or the antihypertensive food composition, and the age, weight, sex, the blood pressure value before ingestion and the like of the subject. And the standard amount of administration (intake) of the CS oligosaccharide per serving or per day can be appropriately set based on non-clinical or clinical test results and the like. Specific examples of the daily dose of the CS oligosaccharide include 10 mg/day or more, 20 mg/day or more, 30 mg/day or more, 40 mg/day or more, 50 mg/day or more, 60 mg/day or more, 70 mg/day or more, 80 mg/day or more, 90 mg/day or more, 100 mg/day or more, and 2000 mg/day or less.

The ingestion period of the CS oligosaccharide can also be appropriately set, but repeated ingestion for a long period of time is preferable. Specific examples of the ingestion period include one week or more, two weeks or more, three weeks or more, four weeks or more, five weeks or more, six weeks or more, seven weeks or more, or eight weeks or more.

The CS oligosaccharide exerts an antihypertensive action, and thus can be used for the purpose of preventing or improving diseases or unhealthy states in which hypertension or a hypertensive state is a factor of the onset or deterioration. The present invention can be used for all applications that have significance in lowering blood pressure.

Examples of the administration target of the CS oligosaccharide include not only humans and animals having a blood pressure value within an optimal or normal range, but also humans (patients) and animals (experimental animals, pet animals, livestock, or the like) that have or may develop hypertension. That is, the present invention also provides prevention or improvement of hypertension, including a step of reducing the blood pressure of a human or animal by administering a chondroitin sulfate oligosaccharide to the human or animal that has developed or may develop hypertension.

Examples of a human who may develop hypertension include a person having a systolic blood pressure in the examination room blood pressure of 130 mmHg or more. More specific examples thereof include a human having blood pressure value being high blood pressure (examination room blood pressure: systolic blood pressure of 130 to 139 mmHg and/or diastolic blood pressure of 85 to 89 mmHg, or home blood pressure: systolic blood pressure of 125 to 134 mmHg and/or diastolic blood pressure of 75 to 84 mmHg) or high blood pressure I (examination room blood pressure: systolic blood pressure of 140 to 159 mmHg and/or diastolic blood pressure of 90 to 99 mmHg, or home blood pressure: systolic blood pressure of 135 to 144 mmHg and/or diastolic blood pressure of 85 to 89 mmHg) (Japanese Society of Hypertension “Hypertension Treatment Guidelines 2019”).

Examples of a human who actually develops hypertension include a person having blood pressure value being second degree hypertension (examination room blood pressure: systolic blood pressure of 160 to 179 mmHg and/or diastolic blood pressure of 100 to 109 mmHg, or home blood pressure: systolic blood pressure of 145 to 159 mmHg and/or diastolic blood pressure of 90 to 99 mmHg), third degree hypertension (examination room blood pressure: systolic blood pressure of 180 mmHg or more and/or diastolic blood pressure of 110 mmHg or more, or home blood pressure: systolic blood pressure of 160 mmHg or more and/or diastolic blood pressure of 100 mmHg or more), or isolated systolic hypertension (examination room blood pressure: systolic blood pressure of 140 mmHg or more and diastolic blood pressure of less than 90 mmHg, or home blood pressure: systolic blood pressure of 135 mmHg or more and diastolic blood pressure of less than 85 mmHg) (Japanese Society of Hypertension “Guidelines for the Treatment of Hypertension 2019”).

Hereinafter, the present invention will be described based on each example. The technical scope of the present invention is not limited to the features shown by these examples.

### EXAMPLES

### (1) Preparation of Food Used in Test

### [1-1] Test Food

280 kg of cartilage of a ray was placed in an oblique shaft kneader, 300 g of papain was added thereto while stirring, and reaction was performed at 55° C. for 3 hours. After deactivation treatment by keeping at 92° C. for 10 minutes, coarse filtration was performed by passing through a wire gauze, and a filtrate was collected. The filtrate was cooled to 50° C., 10 kg of diatomaceous earth was added as a filtration aid, and was well stirred, and then subjected to clarifying filtration using a pressure filtration apparatus, filter press, to provide a pale-yellow clear filtrate. The filtrate was applied to a filtration apparatus equipped with an ultrafiltration membrane having a molecular cutoff of 13000 and dialyzed for 12 hours while appropriately adding water. The internal liquid (dialysis retention liquid) was collected, heat-sterilized at 92° C., and then spray-dried with a spray dryer to provide 10.2 kg of white powder, which was then used as crude purified chondroitin sulfate (polymeric chondroitin sulfate).

The crude purified chondroitin sulfate was hydrolyzed under high temperature and high pressure to provide chondroitin sulfate (CS oligosaccharide) as an oligosaccharide. Specifically, first, an oligosaccharide production apparatus (FIG. 2) described in Japanese Patent No. 6146733 was prepared. That is, as shown in FIG. 2, the apparatus includes a water container that stores distilled water, a high-pressure pump A (Milflo controlled displacement pump M150, pulse-less C24-Z3, Nikkiso Co., Ltd.), a heater (electric heater), a sensor A, a raw material container that stores a raw material, a high-pressure pump B (Milflo controlled displacement pump M150, pulse-less C23-X1, Nikkiso Co., Ltd.), a T-shaped tube for mixing, a reaction unit (made of stainless steel 316 piping (space volume of lumen: 46800 mm³ to 191000 mm³) having a lumen diameter of 0.5 mm), a sensor B, a sensor C, a water bath, a sensor D, a back pressure valve (High Pressure/Back Pressure 26-1762-66-314, TESCOM), and a product container that stores a reaction product, and the water container, the raw material container, and the product container are all connected by a stainless pipe. The inlet temperature and the outlet temperature of the reaction unit are confirmed by the sensor B and the sensor C, respectively. The pressure is confirmed by the sensor D.

10 kg of crude purified chondroitin sulfate was dissolved in 500 L of water and adjusted to Brix 2.0 to provide a raw material solution. The degassed distilled water was placed in a water container, continuously fed by the high-pressure pump A, and heated by a heater. The raw material solution was placed in a raw material container, and continuously fed by the high-pressure pump B. The heated distilled water and the raw material solution at room temperature were mixed in a T-shaped tube for mixing at the inlet of the reaction unit, and water and chondroitin sulfate contained in the raw material solution were reacted in the reaction unit to perform hydrolysis. As reaction conditions, the temperature of the reaction unit was 175 to 220° C., the pressure of the reaction unit was 5 to 25 MPa, and the reaction time was 8.8 seconds. Subsequently, the stainless pipe was directly cooled in a water bath to rapidly terminate the reaction. Thereafter, the pressure in the stainless-steel pipe was reduced by a back pressure valve, and the reaction product was housed in the product container. The flow rate of the distilled water was set to three times or more the flow rate of the raw material solution.

The reaction product was subjected to a filtration apparatus equipped with an ultrafiltration membrane having a molecular cutoff of 3000 to be concentrated, and an internal holding liquid was collected. This was spray-dried with a spray dryer to provide a CS oligosaccharide powder. This was filled into an edible capsule so as to be 50 mg of the CS oligosaccharide per one capsule to prepare a test food.

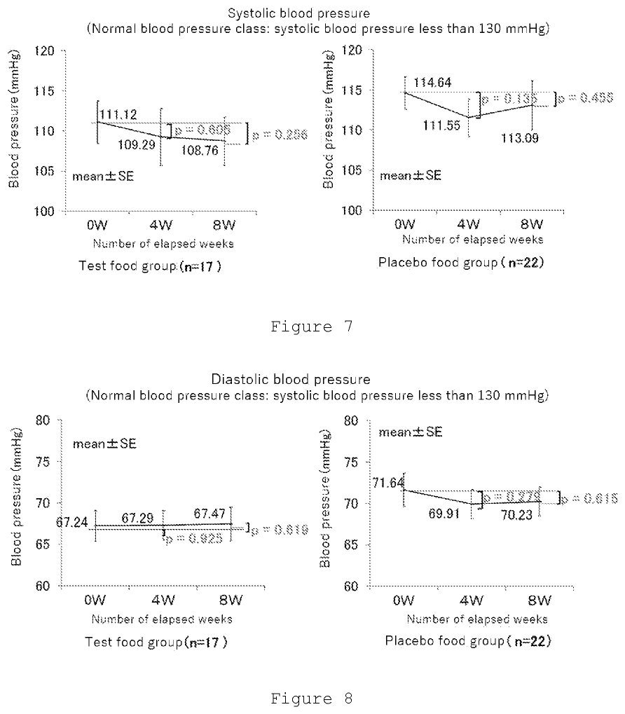

The CS oligosaccharide obtained by the method described in Example (1) [1-1] mainly contains an oligosaccharide having 2 to 12 constituent sugars (Japanese U.S. Pat. No. 6,146,733: paragraphs [0057], [0060], [FIG. 6], and [FIG. 7]). In addition, the CS oligosaccharide is obtained by hydrolysis, and thus has a structure in which the non-reducing end is saturated. In addition, ray cartilage is used as a raw material, and thus chondroitin sulfate C is contained in an amount of 60% or more.

### [1-2] Placebo Food Product

The dry-baked flour was fully filled in an edible capsule to provide a placebo food product.

### [1-3] Nutrient Composition

Nutritional composition analysis of the test food product and the placebo food product (per two capsules) was performed by Japan Food Analysis Center (Chitose, Hokkaido). The results are shown in Table 1. The content of the chondroitin sulfate oligosaccharide was analyzed by MARUKYO SUISAN CO., LTD. using a high-performance liquid chromatography (HPLC) method. The test food product and the placebo food product had completely the same appearance.

TABLE 1

| | Test | Placebo |
|---|---|---|
| | food | food product |
| Calorie (kcal) | 0.32 | 2.14 |
| Water (g) | 0 | 0.07 |
| Protein (g) | 0 | 0.05 |
| Lipid (g) | 0 | 0.01 |
| Carbohydrate (g) | 0.08 | 0.46 |
| Ash (g) | 0.02 | 0 |
| Sodium (mg) | 4.66 | 0.01 |
| Chondroitin sulfate | 100 | 0 |
| oligosaccharide (mg) | | |

### (2) Subject

56 subjects (45 to 68 years old) were stratified by sex, age, and the like, and randomly assigned by computer to a test food group or a placebo food group using stratification block randomization. During the study, physicians, nurses, clinical study coordinators, and statistical analysts were unaware of the allocation information. The characteristics of the test food group and the placebo food group are shown in Table 2 below. As shown in Table 2, there was no significant difference in the numerical values between the groups in each characteristic, and the allocation of the subjects to each group was appropriate.

TABLE 2

| | Test | Placebo | | |
|---|---|---|---|---|
| Properties | food group | food group | p | Test method |
| Number of subjects | 30 | 26 | — | — |
| Number of male | 6 | 5 | 1 | Chi-squared test |
| Age | 57.23 ± 6.67 | 56.85 ± 6.61 | 0.83 | Student's t-test |
| Height (cm) | 157.71 ± 7.51 | 160.48 ± 4.95 | 0.83 | |
| Body weight (kg) | 58.4 ± 11.11 | 56.42 ± 8.29 | 0.12 | |
| Body mass index | 23.37 ± 3.35 | 21.86 ± 2.73 | 0.14 | |
| Ingestion rate (%) | 99.76 ± 1.39 | 99.31 ± 1.90 | 0.94 | Mann-Whitney U-test |

### (3) Ingestion Study (Randomized, Double-Blind, Placebo-Controlled, Parallel-Group Comparative Study)

The test food group was caused to take a capsule of a test food product, and the placebo food group was caused to take a capsule of a placebo food product every day at an intake of two capsules per day. That is, in the test food group, the intake of chondroitin sulfate oligosaccharide was 100 mg/day. The placebo food product had no weight setting for the intake. The ingestion period was 8 weeks. Systolic blood pressure and diastolic blood pressure were measured by a medical worker using an automatic sphygmomanometer HEM-7080IC (OMRON Corporation) before the start of the ingestion period (0 week) and at 4th week and 8th week after the start (blood pressure measured in an examination room).

### (4) Comparison Between Test Food Group and Placebo Food Group

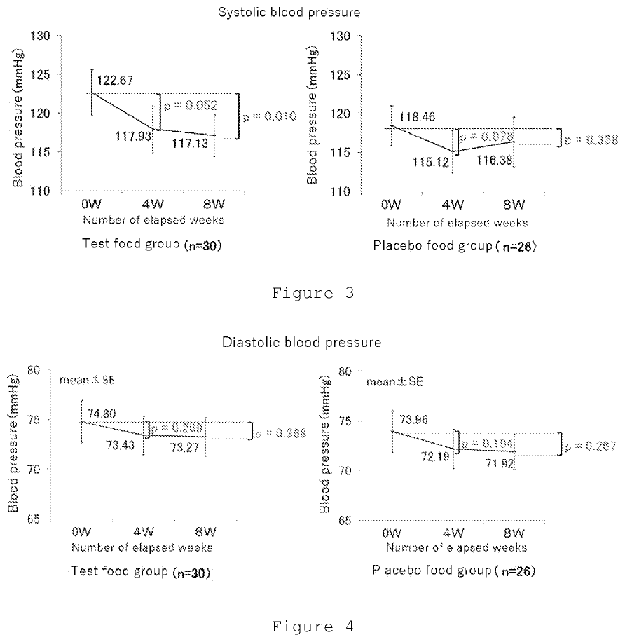

For the blood pressure measured in the present Example (3), the values at 4th week and 8th week with respect to the value at 0 week were tested by Wilcoxon signed rank test, and the p value was calculated for each group of the test food group and the placebo food group. The p value is shown in Table 3, the systolic blood pressure of each group is shown in FIG. 3, and the diastolic blood pressure is shown in FIG. 4.

TABLE 3

| | | Significantly established | |
|---|---|---|---|
| | | for blood pressure at | |
| | | 0 week (p-value) | |
| | | 4th week | 8th week |
| Systolic | Test food group | 0.052 | 0.010 |
| blood pressure | Placebo food group | 0.078 | 0.338 |
| Diastolic | Test food group | 0.269 | 0.368 |
| blood pressure | Placebo food group | 0.194 | 0.267 |

As shown in Table 3 and FIG. 3, in the test food group, systolic blood pressure was significantly reduced 8th week after ingestion (p<0.05) compared to before ingestion (0 week). In contrast, no significant changes were observed in systolic blood pressure in the placebo food group at either the 4th or 8th week. On the other hand, as shown in Table 3 and FIG. 4, no significant changes were observed in diastolic blood pressure in either the test food group or the placebo food group at 4th week and 8th week. That is, subjects who ingested the test food exhibited a significant decrease in systolic blood pressure. These results demonstrated that chondroitin sulfate oligosaccharide has the antihypertensive effect.

### (5) Stratified Analysis

The blood pressure values measured in the present Example (3) were analyzed by dividing into a high blood pressure class and a normal blood pressure class. That is, for each of the test food group and placebo food group, subjects were divided into those with a systolic blood pressure of 130 mmHg or more before the start of the study and those with a systolic blood pressure of less than 130 mmHg, with the former designated as the “high blood pressure class” and the latter designated as the “normal blood pressure class”. In the test food group, 13 people were in the high blood pressure class and 17 people were in the normal blood pressure class. In the placebo food group, there were 4 subjects in the high blood pressure class and 22 subjects in the normal blood pressure class. The measured values at 4th week and 8th week were compared against the measured value at 0 week using the Wilcoxon signed rank test, and p values were calculated for each class in each group.

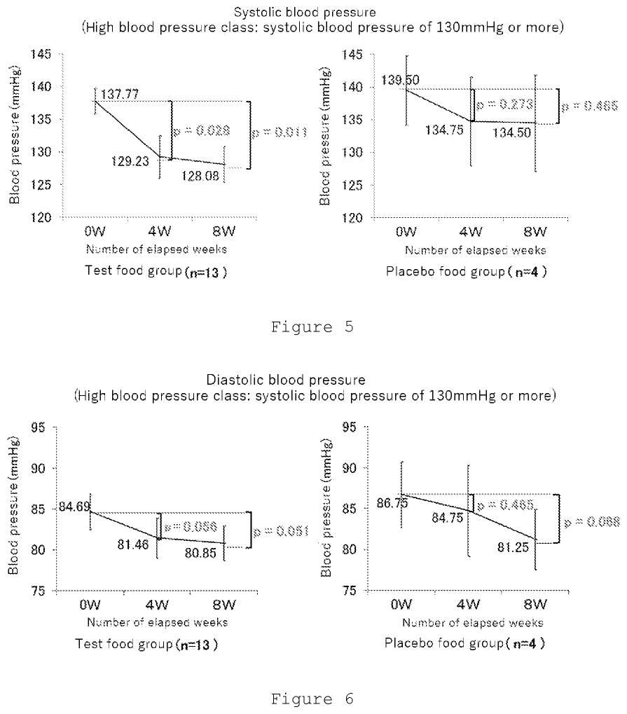

The p-values for the high blood pressure class in each group are shown in Table 4, the systolic blood pressure in FIG. 5, and the diastolic blood pressure in FIG. 6.

TABLE 4

| | | Significantly established | |
|---|---|---|---|
| High blood pressure class | | for blood pressure at | |
| (systolic blood pressure | | 0 week (p-value) | |
| of 130 mmHg or more) | | 4th week | 8th week |
| Systolic | Test food group | 0.028 | 0.011 |
| blood pressure | Placebo food group | 0.273 | 0.465 |
| Diastolic | Test food group | 0.056 | 0.051 |
| blood pressure | Placebo food group | 0.465 | 0.068 |

Test food group n = 13,
Placebo food group n = 4

As shown in Table 4 and FIG. 5, in the high blood pressure class of the test food group, systolic blood pressure decreased significantly (p<0.05) 4th week and 8th week after ingestion compared to before ingestion (0 week). In contrast, in the high blood pressure class of the placebo food group, no significant changes were observed in systolic blood pressure at either 4th week or 8th week. On the other hand, as shown in Table 4 and FIG. 6, no significant changes were observed in diastolic blood pressure at 4th week and 8th week in either the high blood pressure class of the test food group or the placebo food group. That is, in the high blood pressure class (those with systolic blood pressure of 130 mmHg or more) in which the test food was ingested, systolic blood pressure was significantly decreased. These results demonstrated that chondroitin sulfate oligosaccharide has the antihypertensive effect.

Then, the p values for the normal blood pressure class for each group are shown in Table 5, the systolic blood pressure in FIG. 7, and the diastolic blood pressure in FIG. 8.

TABLE 5

| | | Significantly established | |
|---|---|---|---|
| Normal blood pressure class | | for blood pressure at | |
| (systolic blood pressure | | 0 week (p-value) | |
| less than 130 mmHg) | | 4th week | 8th week |
| Systolic | Test food group | 0.605 | 0.256 |
| blood pressure | Placebo food group | 0.135 | 0.455 |
| Diastolic | Test food group | 0.925 | 0.619 |
| blood pressure | Placebo food group | 0.279 | 0.615 |

Test food group n = 17,
Placebo food group n = 22

As shown in Table 5 and FIG. 7, no significant changes were observed in systolic blood pressure at 4th week and 8th week in either the normal blood pressure class of the test food group or the placebo food group. Further, as shown in Table 5 and FIG. 8, no significant changes were observed in diastolic blood pressure at 4th week and 8th week in either the normal blood pressure class of the test food group or the placebo food group. That is, in the normal blood pressure class (those with a systolic blood pressure of less than 130 mmHg) in which the test food was ingested, no significant changes were observed in either systolic or diastolic blood pressure.

These results demonstrated that the chondroitin sulfate oligosaccharide has the effective antihypertensive effect in people with high blood pressure.

1-9. (canceled)

10. A method for lowering a blood pressure or preventing or improving hypertension, comprising administering an effective amount of chondroitin sulfate oligosaccharide to a subject in need thereof, wherein the subject is a human or animal.

11. The method of claim 10 wherein the method is for lowering blood pressure, and the subject is a human or animal in need of lowering blood pressure.

12. The method of claim 10 wherein the method is for preventing or improving hypertension, the subject is suffering from or at risk of developing hypertension, and the administering is to lower the blood pressure of the subject.

13. The method according to claim 10, wherein the blood pressure is systolic blood pressure.

14. The method according to claim 10, wherein the chondroitin sulfate oligosaccharide has 2 to 38 sugar residues as constituent saccharides.

15. The method according to claim 10, wherein the chondroitin sulfate oligosaccharide contains chondroitin sulfate C by 50 mass % or more.

16. The method according to claim 10, wherein the chondroitin sulfate oligosaccharide has a structure in which the non-reducing end is saturated.

17. The method according to claim 10, wherein the subject has a systolic blood pressure of 130 mmHg or more before intake of the chondroitin sulfate oligosaccharide.

18. The method according to claim 10, wherein the effective amount of the chondroitin sulfate oligosaccharide is 100 mg/day or more.

19. The method according to claim 10, wherein the administering is carried out for four weeks or more.

20. The method according to claim 10, wherein the chondroitin sulfate oligosaccharide has chondroitin sulfate C and chondroitin sulfate D with a mass ratio of chondroitin sulfate C:chondroitin sulfate D=50 to 70:1 to 10.

21. The method according to claim 20, wherein the chondroitin sulfate oligosaccharide has chondroitin sulfate C, chondroitin sulfate D, chondroitin sulfate O, and chondroitin sulfate A.

22. The method according to claim 10, wherein the chondroitin sulfate oligosaccharide is derived from cartilaginous fish.
